(19) United States
(12) Patent Application Publication
(10) Pub. No.: US 2025/0235585 A1
(43) Pub. Date: Jul. 24, 2025

(54) Tissue Engineered Spinal Tracts For Functional Regeneration After Spinal Cord Injury

(71) Applicants: THE TRUSTEES OF THE UNIVERSITY OF PENNSYLVANIA, Philadelphia, PA (US); The United States Government as represented by the Department of Veterans Affairs, Washington, DC (US)

(72) Inventors: Daniel Kacy Cullen, Media, PA (US); Justin C. Burrell, Philadelphia, PA (US); Ali K. Ozturk, Wayne, PA (US)

(21) Appl. No.: 18/838,502

(22) PCT Filed: Feb. 18, 2023
(86) PCT No.: PCT/US2023/013385

§ 371 Date: Aug. 14, 2024

Related U.S. Application Data
(60) Provisional application No. 63/311,928, filed on Feb. 18, 2022.

(51) Int. Cl. A61L 27/38; A61L 27/26; A61L 27/36; C07K 14/405; C08L 5/08; C08L 5/12; C08L 89/06; C12N 5/0793; C12N 13/00; C12N 15/86

(52) U.S. Cl. CPC A61L 27/3878; A61L 27/26; A61L 27/3633; A61L 27/3804; C07K 14/405; C08L 5/08; C08L 5/12; C08L 89/06; C12N 5/0619; C12N 13/00; C12N 15/86; A61L 2430/38; C12N 2510/00; C12N 2533/54; C12N 2533/76; C12N 2533/80; C12N 2539/00; C12N 2750/14143

(57) ABSTRACT

The present invention includes a method of fabricating a tissue engineered spinal tract, the method comprising (a) seeding a plurality of optogenetic neural cells at each end of a hydrogel microcolumn to form a construct; and (b) culturing the construct in vitro while stimulating the plurality of optogenetic neural cells with a predetermined wavelength of light.

### CROSS-REFERENCE TO RELATED APPLICATION

The present application is entitled to priority under 35 U.S.C. § 119(e) to U.S. Provisional Patent Application No. 63/311,928 filed Feb. 18, 2022, the content of which is incorporated by reference herein in its entirety.

### STATEMENT REGARDING FEDERALLY SPONSORED RESEARCH OR DEVELOPMENT

This invention was made with government support under NS117757 awarded by the National Institutes of Health and I01 BX003748 awarded by the Department of Veterans Affairs. The government has certain rights in the invention.

### BACKGROUND OF THE INVENTION

Neural regeneration after spinal cord injury (SCI) is hampered by the resulting hostile local microenvironment of the injury. There is a need in the art for structured engineered tissue to facilitate nervous system reconstruction, with prior success using implanted microtissue featuring sensory neurons to span SCI lesions. This disclosure addresses that need.

### SUMMARY

Provided herein, inter alia, is a method of fabricating a tissue engineered spinal tract, the method comprising (a) seeding a plurality of optogenetic neural cells at each end of a hydrogel microcolumn to form a construct; and (b) culturing the construct in vitro while stimulating the plurality of optogenetic neural cells with a predetermined wavelength of light. In some embodiments, the plurality of optogenetic neural cells are transduced with one or more transgenes. In some embodiments, the one or more transgenes include channelrhodopsin, Channelrhodopsin-2, ChrimsonR, CatCh, a halorhodopsin, a archaerhodopsin, an optogenetic sensor for calcium, an optogenetic sensor for chloride, and/or an optogenetic sensor for membrane voltage. In some embodiments, the one or more transgenes include channelrhodopsin, Channelrhodopsin-2, ChrimsonR, CatCh, a halorhodopsin, and/or a archaerhodopsin. In some embodiments, the optogenetic sensors for calcium include Aequorin, Cameleon, or GCaMP. In some embodiments, the optogenetic sensors for chloride include clomeleon. In some embodiments, the optogenetic sensors for membrane voltage include Mermaid. In some embodiments, the plurality of optogenetic neural cells are transduced using AAV to insert ChrimsonR with a human synapsin promoter.

In some embodiments, the construct is a biocompatible construct. In some embodiments, the construct is an implantable construct. In some embodiments, the method further comprises (c) determining axons growth from the plurality of neural cells has reached a particular length; and (d) responsive to the particular length of axon growth being determined to have been reached, packaging and/or providing the micro-column for implantation. In some embodiments, the particular length is a predetermined desired length. In some embodiments, the particular length ranges from about 0.5 to about 5 centimeters. In some embodiments, the particular length ranges is about 1.2 centimeters. In some embodiments, step (c) comprises imaging the micro-columns and neural cells therein.

In some embodiments, the plurality of optogenetic neural cells with which the micro-column is seeded at step (a) comprise a population of neural cells. In some embodiments, the population of neural cells is seeded individually, as an organoid, or as an aggregate. In some embodiments, the neural cell aggregates comprise a plurality of approximately spherical aggregates of neural cells. In some embodiments, each neural cell aggregate comprises cells at a density ranging from about 10,000 to about 3,000,000 neurons per aggregate. In some embodiments, each neural aggregate comprises cells at a density ranging from about 40,000 to about 65,000 motor neurons per aggregate. In some embodiments, a plurality of the neural cell aggregates exhibit a diameter of between 10 μm to 2500 μm. In some embodiments, the diameter of the neural cell aggregate is about 500 μm.

In some embodiments, the micro-column comprises a hydrogel sheath and a core comprising an extracellular matrix (ECM), and wherein the neural cells are seeded to be in direct contact with the ECM of the core. In some embodiments, the hydrogel includes agarose, gelatin, silk, chitosan, hyaluronic acid, methylacrylated gelatin, methylacrylated hyaluronic acid (MeHA), or combinations thereof. In some embodiments, the hydrogel sheath comprises MeHA. In some embodiments, the hydrogel sheath comprises agarose and gelatin.

Also provided herein, in some embodiments, is a method of treating a spinal cord injury in a subject, comprising contacting a lesion in the spine of the subject with a tissue engineered spinal tract made by the processes disclosed herein.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1C Schematic of Chronic Spinal Cord Injury (SCI) Pathology and Pathway Reconstruction Using a Tissue Engineered Spinal Tract (TE-ST). (A) Illustration of the injured spinal cord following balloon-compression injury. Reactive glial cells (astrocytes, microglia, macrophages) proliferate and form an inhibitory hostile environment that encapsulates the necrotic tissue. (B) An illustration depicting the surgical approach and TE-ST implantation at two weeks post-SCI. (C) After transplantation, surviving motor neurons and axons within the protective agarose hydrogel as well as axons extending into the host spinal cord parenchyma can be visualized based on endogenous expression of TdTomato (red).

FIGS. 2A-2E Motor Neuron Tissue Engineered Spinal Tract (“Motor TE-ST”) Fabrication and Characterization. TE-STs are anatomically-inspired three-dimensional engineered micro-tissue containing a discrete motor neuron population on the ends of an agarose microcolumn. TE-STs can be fabricated at various lengths depending on the application. (A-B) Representative images showing robust healthy neurons with axons spanning the lumen of the column via (A) phase microscopy at 3 days in vitro (DIV) and (B) confocal microscopy following a live-dead assay at 5 DIV with green showing surviving cells and red labeling dead cells. (C) Representative confocal imaging of TE-STs at 7 DIV. Immunocytochemistry using choline acetyltransferase (ChAT) and beta-III-tubulin (Tuj1) to discriminate neuronal phenotype confirmed the localization of two discrete populations of ChAT+ motor neurons with Tuj1+ axons extending the length of the micro-column. Motor neurons were transduced to express GFP to aid in visualization. High resolution confocal imaging revealed discrete regions of Tuj-1+/ChAT+/GFP+ motor neurons and axons, with Hoechst (a nuclei marker) counterstain. (D-E) Representative confocal images of motor TE-STs at 3 mm (D) and 6 mm (E) are shown with motor neurons transduced to express TdTomato (red) co-labeled with Tuj-1 (neuronal/axon marker). Scale bars: (A-E) 100 μm.

FIGS. 3A-3C High-Resolution Imaging of Motor TE-ST Axons. TE-ST containing motor neurons expressing TdTomato (red) were fixed at 21 DIV and then labeled for Tuj1 (neuronal/axon marker; green). The transparent agarose hydrogel enabled visualization of the motor axons at high resolution using a light sheet microscope. (A-C) Robust axonal networks can be readily seen throughout the entire lumen of the micro-column co-labeling for TdTomato and Tuj1. The light sheet microscope acquires images from two objectives and then the software digitally “fuses” the image, which allows greater detail and localization of the fine processes as compared to conventional microscopic imaging. While some axons extended through the collagenous ECM core, a substantial portion grew in a corkscrew fashion on the surface of the ECM at the interface with the agarose micro-column.

FIGS. 4A-4D TE-ST Fabrication at Scales Necessary for Pathway Reconstruction and Evidence of Graft Survival At 1 Month Following Implantation Across a T6/T7 SCI. (A) Immunocytochemistry of a 1.2 cm long motor TE-ST at 21 DIV. Motor TE-ST comprised of two populations of motor neurons transduced to endogenously express TdTomato (red) to aid visualization. Neurons and axons were labeled with Tuj1 (far red). (B) TE-STs were grown in vitro for 21 days to allow neurite outgrowth across the 1.2 cm inner lumen prior to implantation. Phase microscopy was performed prior to all implants to qualitatively assess neuronal health and to evaluate axonal cytoarchitecture pre-implantation. Scale: 500 μm. (C) Schematic demonstrating motor TE-ST implantation and potential neuronal relay mechanism. (D) TE-ST visualized at 1 week post-implantation following tissue clearing and multiphoton imaging. Healthy implanted motor neurons were found within the TE-ST indicating the protective hydrogel outer encasement enabled survival following implantation within the hostile host spinal cord environment. Discrete regions of neurons and axons were found within the lumen, indicating the engineered neuronal-axonal cytoarchitecture was maintained following implantation. Scale: 100 μm.

FIGS. 5A-5C Motor TE-ST Survival Up to 3 Months Post-Transplantation in a Rodent Model of SCI. (A) At 3 months post-transplantation, spinal cord tissue was optically cleared and surviving neurons were readily visualized within the lumen of the micro-column. Three-dimensional volumetric reconstruction of the TE-ST shows neuronal survival at 3 months and evidence of integration with the host spinal cord. (B-C) Individual z-planes are shown to highlight surviving (B) motor neurons and (C) their axon tracts.

FIG. 6 Schematic Depicting Proposed Mechanisms for Spinal Cord Pathway Reconstruction using TE-STs. (Top) Proposed Mechanism 1: Sensory axons extending from a sensory TE-ST structurally integrate with the native host tissue and serve as a “living scaffold” for rapid host axonal extension across injured spinal cord. (Middle) Proposed Mechanism 2: Motor axons extending a motor TE-ST synaptically integrate with the native host neurons rostral and caudal to the injury site to act as a neuronal relay and restore connectivity across the damaged spinal cord. (Bottom) Proposed Mechanism 3: TE-STs comprised of both motor and sensory neurons may simultaneously exploit a combination of both of these mechanisms and rapidly accelerate host axons across the graft and restore functional connectivity via a neuronal relay.

FIG. 7 shows images illustrating slowing of motor neurite growth in vitro. The images show unstimulated motor neurons at 8 days in vitro (DIV; top), 11 DIV (middle), and 13 DIV (bottom).

FIG. 8 shows a schematic illustrating optogenetic stimulation for enhanced axonal growth during neuronal construct fabrication.

FIGS. 9A-C show images and a graph illustrating optogenetic stimulation of three-dimensional motor neurons. (A) Schematic illustrating the optogenetic stimulation paradigm. (B) Confocal reconstruction at 21 DIV across the different groups. Immunohistochemistry was performed to identify axons (Tuj1, green). (C) Quantification of neurite length across the different groups. Two-way ANOVA was performed with transduction and stimulation as the independent variables and the neurite length as the dependent variable. *p<0.05, **p<0.01, ***p<0.001, ****p<0.0001. Scale bars: 500 μm.

FIGS. 10A-10B show an image and a graph illustrating the effects of different frequencies on motor neuron (MN) aggregates during optogenetic stimulation. (A) Immunohistochemistry of a fixed culture, following stimulation, showing axons labeled with Tuj1 (purple). (B) Quantification of total area of neurite outgrowth for various frequencies.

FIG. 11 shows a graph illustrating engineered spinal motor neurite growth following light stimulation.

FIGS. 12A-12C show images and graphs illustrating the effects of different vector genome per cell (vg/cell) levels on human motor neuron transduction. (A) Immunohistochemistry of various vg/cell levels. (B) Differences in total number of cells labeled with hoechst nuclear marker (HST) at 12 days in vitro. (C) Quantification of transduced neurons labeled with anti-red fluorescent protein (RFP) following transduction at different vg/cell levels.

FIGS. 13A-13B show images and a graph illustrating the effects of different viral dosing levels on human motor neuron aggregate transduction. (A) Fluorescence at various viral dosing levels. (B) Quantification of total fluorescence measured from the aggregate (CTAF) for various viral dosing levels.

FIGS. 14A-14C show images illustrating engineered spinal tracts following spinal cord injury. (A) Low magnification imaging showing the engineered axonal cytoarchitecture in the protective agarose microcolumn spanning the injury site. Neurons are visible at the end the microcolumn. (B) High resolution imaging demonstrating neurite outgrowth into spinal cord parenchymal tissue. (C) Axial rendering of the micro-column showing the bundle of axons in the center of the lumen protected by the agarose micro-column.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the singular form “a,” “an,” and “the” include plural references unless the context clearly dictates otherwise.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

As used herein, the term “cylinder” or “cylindrical” includes a surface consisting of each of the straight lines that are parallel to a given straight line and pass through a given curve. In some embodiments, cylinders have an annular profile. In other embodiments, the cylinder has a cross-section selected from the group consisting of: a square, a rectangle, a triangle, an oval, a polygon, a parallelogram, a rhombus, an annulus, a crescent, a semicircle, an ellipse, a super ellipse, a deltoid, and the like. In other embodiments, the cylinder is the starting point of a more complex three-dimensional structure that can include, for example, complex involutions, spirals, branching patterns, multiple tubular conduits, and any number of geometries that can be implemented in computer-aided design, 3-D printing, and/or in directed evolutionary approaches of secretory organisms (e.g., coral), including of various fractal orders.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit.

“Forced aggregation” and “forced cell aggregation” are used interchangeably herein, and refer to a method of forming “aggregates” or “spheres” of neurons by centrifugation in inverted pyramidal micro-wells.

By the term “modified” as used herein, is meant a changed state or structure of a molecule or cell of the invention. Molecules may be modified in many ways, including chemically, structurally, and functionally. Cells may be modified through the introduction of nucleic acids.

As used herein, the term, “optogenetic neuron” refers to neurons that have been genetically modified to express light-sensitive ion channels. For example, the neurons can express one or more optogenetic actuators such as channelrhodopsin, halorhodopsin, and archaerhodopsin and/or one or more optogenetic sensors for calcium (e.g., Aequorin, Cameleon, GCaMP), chloride (e.g., Clomeleon) or membrane voltage (e.g., Mermaid). Further details are found in PCT application No. PCT/US2017/027705 which is hereby incorporated by reference.

Unless specifically stated or obvious from context, the term “or,” as used herein, is understood to be inclusive.

A “subject” or “patient,” as used therein, may be a human or non-human mammal. Non-human mammals include, for example, livestock and pets, such as ovine, bovine, porcine, canine, feline and murine mammals. Preferably, the subject is human.

The term “therapeutic” as used herein means a treatment and/or prophylaxis. A therapeutic effect is obtained by suppression, remission, or eradication of a disease state.

To “treat” a disease as the term is used herein, means to reduce the frequency or severity of at least one sign or symptom of a disease or disorder experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention includes compositions and methods for neuronal growth and implantation. Without meaning to be limited by theory, the invention is based in part on the discovery that stimulation of optogenetic neural cells with light while growing the cells in vitro, as shown in the various figures and examples herein, produces faster more robust growth relative to non-transduced or transduced but non-stimulated controls. Accordingly, in some embodiments, the method includes generating an optogenetic neural cell, seeding the optogenetic neural cell in a column to form a construct, culturing the construct in vitro, and stimulating the optogenetic neural cell with a predetermined wavelength of light during the culturing.

A skilled person will appreciate that optogenetic neurons may be generated in various ways from any suitable neural cell. Suitable neural cells include, but are not limited to, motor neurons, sensory neurons, interneurons, cortical, subcortical, cerebellar, spinal, propriospinal, cholinergic, dopaminergic, gamma-aminobutyric acid (GABA), glutamatergic, histaminergic, noradrenergic, serotonergic, or combinations thereof. These neural cells may be embryonic, autogenic, allogenic, or xenogenic. In some embodiments, the neural cells are isolated from transgenetic animals. For example, in some embodiments, the neural cells include embryonic motor neurons or human iPSC-derived motor neurons. Suitable methods for generating optogenetic neurons include, but are not limited to, viral transduction (AAV, lentivirus), electroporation, CRE recombinase, neuron isolation of genetically modified animals, CRISPR-Cas9, or lipid nanoparticle delivery. In some embodiments, for example, neurons may be transduced with a transgene to generate optogenetic neurons sensitive to light-activation of ion channels or g-coupled mediated protein signaling.

A variety of transgenes may be used to form the optogenetic neurons and all of them are intended to be encompassed by the present invention. For example, in various embodiments, the optogenetic neural cells are transduced with channelrhodopsin. Other suitable transgenes include, but are not limited to, Channelrhodopsin-2, ChrimsonR, CatCh, halorhodopsins, archaerhodopsins, one or more optogenetic sensors for calcium (e.g., Aequorin, Cameleon, GCaMP), one or more optogenetic sensors for chloride (e.g., Clomeleon), and/or one or more optogenetic sensors for membrane voltage (e.g., Mermaid). For example, in some embodiments, the optogenetic neural cells are transduced with channelrhodopsin, Channelrhodopsin-2, ChrimsonR, CatCh, halorhodopsins, and/or archaerhodopsins. Cell-specific transduction may be achieved by placing the desired transgene under control of a promoter sequence. For example, synapsin, CAG, cytomegalovirus (CMV), elongation factor 1α, and/or α-calcium/calmodulin-dependent kinase II (aCamKII).

The column includes any suitable column for permitting light stimulation of the optogenetic neurons seeded therein, such as, but not limited to, a transparent micro-column. In some embodiments, the transparent micro-column permits both light stimulation through the column as well as imaging over time (e.g., phase microscopy). In some embodiments, the column includes a hydrogel sheath with a core for receiving the optogenetic neural cell therein. In some embodiments, the hydrogel sheath comprises a transparent agarose. Other suitable column materials include, but are not limited to, other hydrogels, including gelatin, silk, chitosan, hyaluronic acid, methylacrylated gelatin, methylacrylated hyaluronic acid, or any combination with or without agarose. For example, in some embodiments, the hydrogel sheath comprises MeHA or agarose and gelatin. The core includes any suitable composition for receiving the optogenetic neural cell. For example, in some embodiments, the core includes an extracellular matrix (ECM) arranged and disposed to contact the optogenetic neural cell directly. Additionally or alternatively, the core may include collagen, laminin, fibronectin, Matrigel, Geltrex, or any other combination of proteins. For example, in some embodiments, the micro-column comprises a hydrogel sheath and a core comprising an ECM, where the neural cells are seeded to be in direct contact with the ECM of the core.

In some embodiments, one or more optogenetic neural cells are seeded at one end of the column. Alternatively, one or more neural cells may be seeded at both ends of the column. In such embodiments, the neural cells at either or both ends of the column may be optogenetically transduced. In some embodiments, one or more optogenetic neural cells may be transduced before or after construct fabrication. The one or more optogenetic neural cells may be seeded individually (e.g., as dorsal root ganglia explants), as an organoid (e.g., grown in culture as an organoid), or as an aggregate. For example, in some embodiments, a plurality of optogenetic neural cells are seeded at one or both ends of the column as one or more forced aggregate (i.e., aggregates generated by centrifugation). In some embodiments, the construct is seeded with neural cell aggregates comprising a plurality of approximately spherical aggregates of neural cells. In some embodiments, each neural cell aggregate comprises cells at a density ranging from about 10,000 to about 3,000,000 neurons per aggregate. For example, each neural aggregate may comprise cells at a density ranging from about 40,000 to about 65,000 motor neurons per aggregate. Additionally or alternatively, in some embodiments, a plurality of the neural cell aggregates exhibit a diameter of a diameter of at least 500 μm, between 10 μm to 2500 μm, or any combination, sub-combination, range, or sub-range thereof. For example, the neural aggregate may include a diameter of about 500 μm.

As will be appreciated by those skilled in the art, optogenetic neurons generated in different ways may be stimulated by various wavelengths of light. Accordingly, the wavelength of light is selected to be a wavelength that will stimulate the optogenetic neurons seeded within the column based on the transgene. For example, optogenetic neurons may be sensitive to light with a wavelength ranging between 1-2500 nm. A user skilled in the art will appreciate optogenetic neurons can be activated with light in a number of ways, including white light, light-emitting diode, laser, chemogenetics, upconversion nano/micro particles, or any combination of these modalities. Additionally, in some embodiments, light-sensitive constructs may be stimulated in vitro during the fabrication process or in vivo following transplantation to promote neurite outgrowth and integration. FSpecific cell types may be more sensitive to a specific frequency of light stimulation. For example, in some embodiments, the transgene includes channelrhodopsin and the frequency is between 1 and 130 Hz, between 1 and 100 Hz, between 1 and 75 Hz, between 1 and 50 Hz, between 1 and 20 Hz, between 5 and 130 Hz, between 5 and 100 Hz, between 5 and 75 Hz, between 5 and 50 Hz, between 5 and 20 Hz, about 5 Hz, about 10 Hz, about 20 Hz, or any combination, sub-combination, range, or sub-range thereof. In one embodiment, the transgene includes ChrimsonR and the frequency is between 5 and 20 Hz. In another embodiment, the transgene includes ChrimsonR and the frequency is about 10 Hz.

The optogenetic neurons may also be stimulated at any suitable time point and/or for any suitable length of time. Suitable lengths of time include, but are not limited to, up to 2 hours, up to 1.5 hours, up to 1 hour, between 15 minutes and 2 hours, between 15 minutes and 1.5 hours, between 30 minutes and 1.5 hours, about 1 hour, or any combination, sub-combination, range, or sub-range thereof. Additionally, the optogenetic neurons may be stimulated at any time point following optogenetic transduction. As will be appreciated by those skilled in the art, the time for optogenetic transduction may differ between different cells (e.g., up to 4 days for cells not from a transgenic animal). Therefore, depending upon the cell and time for optogenetic transduction, suitable time points may include, but are not limited to, at least 1 day in vitro, between 1 and 14 days in vitro, between 1 and 10 days in vitro, between 1 and 7 days in vitro, 1 day in vitro, 4 days in vitro, 7 days in vitro, 10 days in vitro, or any combination, sub-combination, range, or sub-range thereof. In some embodiments, optogenetic neurons may be stimulated multiple times during the fabrication process (e.g., every other day, once at day 7 and once at day 10).

In various embodiments, the construct is a biocompatible construct. In various embodiments, the construct is an implantable construct. In various embodiments, the method further includes determining axons growth from the plurality of neural cells has reached a particular length; and responsive to the particular length of axon growth being determined to have been reached, packaging and/or providing the micro-column for implantation. In various embodiments, the particular length is a predetermined desired length. In various embodiments, the particular length ranges from about 0.5 to about 5 centimeters, about 0.5 to about 4 centimeters, about 0.5 to about 3 centimeters, about 1 to about 5 centimeters, about 1 to about 4 centimeters, about 1 to about 3 centimeters, about 1 to about 2 centimeters, about 1 centimeter, about 1.2 centimeters, about 1.5 centimeters, about 2 centimeters, about 3 centimeters, about 4 centimeters, about 5 centimeters, or any combination, sub-combination, range, or sub-range thereof. In various embodiments, the particular length ranges is about 1.2 centimeters. In various embodiments, the step of determining axon growth comprises imaging the micro-columns and neural cells therein.

Without meaning to limit the compositions to uses only in a specific method, the constructs fabricated by this method may be useful for the treatment of spinal cord injuries. Accordingly, in one aspect, the method of fabricating a tissue engineered spinal tract includes seeding a plurality of optogenetic neural cells at one or both ends of a hydrogel microcolumn to form a construct; and culturing the construct in vitro while stimulating the plurality of optogenetic neural cells with a predetermined wavelength of light. In another aspect, a method of treating a spinal cord injury in a subject includes contacting a lesion in the spine of the subject with the tissue engineered spinal tract made by the processes disclosed herein. Additionally or alternatively, in some embodiments, prior to contacting the lesion, the method includes determining axons growth from the plurality of neural cells has reached a particular length; and responsive to the particular length of axon growth being determined to have been reached, packaging and/or providing the micro-column for implantation. In some embodiments, optogenetic stimulation may be used post transplantation to enable rapid growth in vivo (e.g., to babysit the distal nerve pathway and muscle).

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1

### Methods and Materials

The materials and methods employed in this Example are now described.

### Embryonic Neuron Isolation and Spinal Motor Aggregation

Spinal cords were isolated from embryonic day 16 Sprague-Dawley rats (Charles River, Wilmington, MA) as previously described. Embryonic spinal motor neurons (MNs) were isolated from dissociated spinal cords using an Optiprep density gradient, plated in a polydimethyl siloxane reverse pyramid (12 μL of 100,000 dissociated MNs), and then centrifuged at 1500 RPM for 5 minutes. Aggregated MNs were transduced overnight in media with AAV2/1.hSynapsin.EGFP.WPRE.bGH or AAV2.hSynapsin.tdTomato vector (UPenn Vector Core). Spinal astrocyte-conditioned neurobasal media with 10% FBS was supplemented with 37 ng/ml hydrocortisone, 2.2 μg/mL isobutylmethylxanthine, 10 ng/ml BDNF, 10 ng/ml CNTF, 10 ng/mL CT-1, 10 ng/ml GDNF, 2% B-27, 20 ng/mL NGF, 20 μM mitotic inhibitors, 2 mM L-glutamine, 417 ng/mL forskolin, 1 mM sodium pyruvate, 0.1 mM β-mercaptoethanol, 2.5 g/L glucose.

### Tissue Engineered Spinal Tracts (TE-STs) Fabrication

TE-STs were constructed using methodology similar to what has previously described been described to fabricate other types of micro-tissue engineered constructs. Briefly, TE-STs were grown by seeding neuronal aggregates at the ends of a cylindrical agarose hydrogel outer encasement with an extracellular matrix inner lumen. Briefly, the outer hydrogel encasement was generated by drawing molten 3% agarose into a capillary tube (701 μm inner diameter). Prior to adding molten agarose, an acupuncture needle (300 μm diameter) was inserted into the glass capillary tube to produce the inner lumen. Unless otherwise stated, micro-columns were 12 mm long with a 701 μm outer diameter and 300 μm inner diameter. Micro-columns were stored in Dulbecco's phosphate-buffered saline at 4° C. Micro-columns were UV-sterilized before approximately 25 μm of extracellular matrix containing rat tail Type 1 collagen (1.0 mg/ml) and laminin (0.1 mg/ml) in 11.70 mM N-(3-Dimethylaminopropyl)-N′-ethyl carbodiimide hydrochloride, 4.3 mM N-Hydroxy succinimide, and 35.6 mM sodium phosphate monobasic was added to each end of column. Extracellular matrix was allowed to polymerize in the incubator for 30 minutes before being rinsed three times with PBS, and then micro-columns were returned to the incubator overnight. DRG explants or motor neuron aggregates were seeded on both ends of the microcolumn to fabricate sensory TE-STs or motor TE-STs, respectively. TE-STs returned to the incubator and allowed to grow with half changes every other day. Approximately n=15-20 TE-STs were fabricated for in vitro characterization and n=20 TE-STs for transplantation. Of the TE-STs generated for transplantation, constructs were only implanted if deemed healthy. Neuronal health was assessed via phase microscopy starting at 4 days in vitro (DIV) and then checked every other day prior to transplantation at 21 DIV.

### Live-Dead Viability Assay

To assess cell viability, media was aspirated and TE-ST cultures were incubated for 30 minutes with 10 mL of Dulbecco's phosphate buffered saline (DPBS) containing calcein AM (5 μl; 4 mM in anhydrous dimethyl sulfoxide) and ethidium homodimer-1 (EthD-1; 20 μl; 2 mM in DMSO/H₂O 1:4 v/v). After incubation, the solution was removed and cultures were rinsed 2-3 times with DPBS. Samples were immediately imaged in DPBS using confocal microscopy.

### Immunocytochemistry

TE-STs were fixed at various time points (N=12 across time points spanning 1-21 DIV) with 4% paraformaldehyde for 35 minutes, rinsed in 1×PBS, permeabilized with 0.3% Triton X100+4% horse serum in PBS for 60 minutes, and then incubated with primary antibodies overnight at 4° C. Primary antibodies were Tuj-1/beta-III tubulin (T8578, 1:500, Sigma-Aldrich) to label axons and choline acetyltransferase (ChAT, ab18376, 1:500, Abcam) to label motor neurons. Following primary antibody incubation, TE-STs were rinsed in PBS and incubated with fluorescently-tagged secondary antibodies (1:500; Invitrogen) for 2 h at 18°-24° C. Finally, Hoechst (33342, 1:10,000, ThermoFisher) was added for 10 min at 18°-24° C. before rinsing in PBS.

### Epidural Balloon-Compression SCI Rodent Model

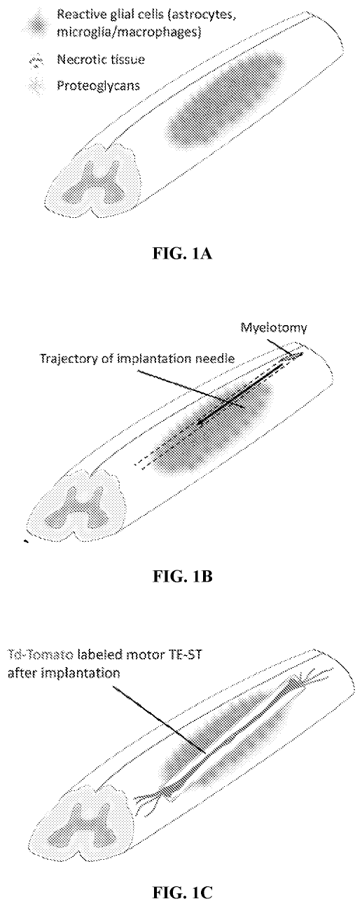

Animals were randomly enrolled into a treatment group at 14 days following an epidural T6/T7 balloon-compression SCI as previously described. Briefly, the animals were anesthetized with isoflurane (induction: 5%, maintenance: 2-3%) and the dorsal aspect of the lower back was shaved and cleaned with betadine solution. A 2 cm midline skin incision was made centered at the rostral margin of the iliac crest to approximate L3-4. After exposing the L3-4 spinous process, the lamina was removed using a bone rongeur until the cauda equina was visualized. A 2F Fogarty catheter was introduced into the epidural space and advanced rostrally within the spinal canal 5 cm to the mid thoracic spine (FIG. 1A). Compression injury was completed by inflating the balloon with 0.6 mL of air for 5 minutes under constant pressure. Correct placement of the epidural balloon was confirmed by visualizing the hindlimb and tail twitching after inflation. Next, the deflated balloon and catheter was removed from the L3-4 space. The incision was stapled closed and animals were allowed to recover before returning to the vivarium. Postoperatively, Cefazolin was administered prophylactically and subcutaneously (30 mg/kg) and Buprenorphine-SR was administered subcutaneously (1 mg/kg every 24-48 hours for 3 days). Subcutaneous 0.9% normal saline was also administered for five days postoperatively. Bladder function was monitored via manual expression to evaluate for restoration of continence. Bladder size was rated as small, medium or large. Bladders were manually expressed twice a day until bladder size is rated as small for 48 consecutive hours, indicating restoration of bladder continence.

### TE-ST Transplantation

A total of 18 animals were randomly enrolled into experimental groups for this study (n=11 cellular and n=7 acellular). Experimental animals underwent a second surgery at day 14 post-injury. TE-ST neuronal health was confirmed pre-transplantation at 21 DIV. Animals were anesthetized and prepared for surgery as previously described. A 3 cm midline dorsal incision was made centered at T7 (defined as the area 5 cm rostral to the L3-4 iliac crest line). A bone rongeur was used to remove the spinous process and lamina at T7, as well as partial T6 and T8 laminectomies to exposure the region of spinal cord compression. A myelotomy was then created using an arachnoid knife lateral to the dorsal spinal vein and an absorbable gelatin sponge was utilized for hemostasis. At 14 days in vitro, TE-STs were loaded into a custom-fabricated blunt needle and prepared for implantation. A 50 μL Hamilton syringe, affixed with the blunt needle loaded with a TE-ST and plunger, was advanced via stereotactic device into the surgical myelotomy site and the TE-ST was deposited into the parenchyma of the spinal cord (FIG. 1B). After transplantation, the needle was withdrawn, and the surgical site closed (FIG. 1C). The terminal time points for this study were 1 week (n=2 cellular), 1 month (n=5 cellular and n=4 acellular), and 3 months (n=4 cellular and n=2 acellular). One animal was humanely euthanized early at 2 months and was excluded from the study.

### Immunohistochemistry

At the terminal time point, animals were euthanized via transcardial perfusion of heparinized saline followed by 10% formalin. Spines were postfixed overnight at 4° C. and then spinal cords were harvested. Spinal cords were tissue clearing by adapting a previously established Visikol protocol. Briefly, spinal cords were rinsed overnight with PBS at 4° C., dehydrated in a series of ethanol washes for 2 hours each (30%, 50%, 70%, and 90%) and 100% ethanol for 24 hours. Next, nerves were incubated in Visikol 1 for 24 hours followed by Visikol 2 for at least 24 hours to complete the clearing process.

### Data Analyses and Imaging

Phase-contrast microscopy images of TE-STs were taken over several days in vitro (DIV). TE-ST viability and presence of the desired neuronal phenotype(s) was qualitatively assessed at 10× magnification using a Nikon Eclipse Ti-S microscope, paired with a QIClick camera and NIS Elements BR 4.13.00. Confocal imaging of TE-STs were taken on a Nikon AIRSI Laser Scanning confocal microscope paired with NIS Elements AR 4.50.00 using a 10× air objective, 16× water immersion objective, or 60× oil objective (1024×1024 pixels, Nikon NIS-Elements AR 3.1.0, Nikon Instruments, Tokyo, Japan). Sequential slices of 10-20 μm in the z-plane were acquired for each fluorescent channel. All confocal images presented are maximum intensity projections of the confocal z-slices. High resolution imaging of TE-STs was also achieved using light sheet microscopy (Bruker MuVi SPIM LS). A light sheet was formed by light emitted by two orthogonal 10× objectives and fluorescent light was captured from two orthogonal 20× objectives (Nikon). Images were processed for three dimensional visualization (Bruker Luxendo). TE-ST survival was assessed by imaging cleared spinal cords and visualized with a multiphoton microscope (Nikon).

### Motor Neuron Aggregation & Transduction.

Motor neurons (MNs) aggregates were generated by centrifuging a solution of dissociated embryonic rodent spinal motor neurons in inverted pyramid wells to create “forced aggregates”0.5, 6 MNs were transduced with chrimsonR (AAV1.Syn.ChrimsonR-tdTomato.WPRE.bGH) overnight immediately after aggregation to enable optogenetic stimulation (light-mediated activation) and were compared to non-transduced groups that served as a control.

### Micro-Column Fabrication.

Agarose hydrogels micro-columns were constructed using a three-phase process similar to methods previously described. Briefly, agarose micro-columns were formed using glass capillary tubes (345-701 μm) allowing for the insertion of acupuncture needles (180-350 μm) through the lumen. Molten agarose (3% weight/volume) in Dulbecco's phosphate buffered saline (DPBS) was added to the capillary tube containing the acupuncture needle and allowed the cool. The acupuncture needle was quickly removed to create the hydrogel shell, and the micro-columns were stored in DPBS at 4° C. Agarose-gelatin micro-columns (1.5% agarose+1.5% gelatin) were fabricated as described above except that micro-columns were stored in 7 mL DPBS with 100 μL at room temperature overnight and subsequently washed 3 times in DPBS prior to further experiments. All micro-columns were cut to the appropriate length, UV sterilized for 30 minutes, and stored in DPBS at 4° C.

Micro-columns were transferred to a new petri dish and excess DPBS was removed from the lumen of the micro-column via micropipette and replaced by extracellular matrix (ECM), comprised of 1.0 mg/ml rat tail collagen+1.0 mg/ml mouse laminin (Reagent Proteins, San Diego, CA). DRG explants or motor neuron aggregates were carefully placed at the ends of the micro-columns containing ECM, under stereoscopic magnification using fine forceps and were allowed to adhere for 45 min at 37° C., 5% CO2. Motor TE-STs were created by seeding a motor neuron aggregate on each end of a micro-column.

### Optical Stimulation Paradigm.

To optimize a stimulation paradigm for motor neurons, neurons cultured in 2D were stimulated with red light at 4 days in vitro (4 DIV) using a custom-made Arduino-powered LED at various frequencies with a one second pulse duration for one hour. Each well was treated to its specified frequency treatment, and a row of unlit LEDs were encoded surrounding each well to minimize light interference into nearby wells. This was translated to a 3D environment following stimulation optimization, where micro-TENGs were stimulated at 7 DIV for one hour and fixed at 21 DIV.

### Results

### TE-ST Characterization In Vitro

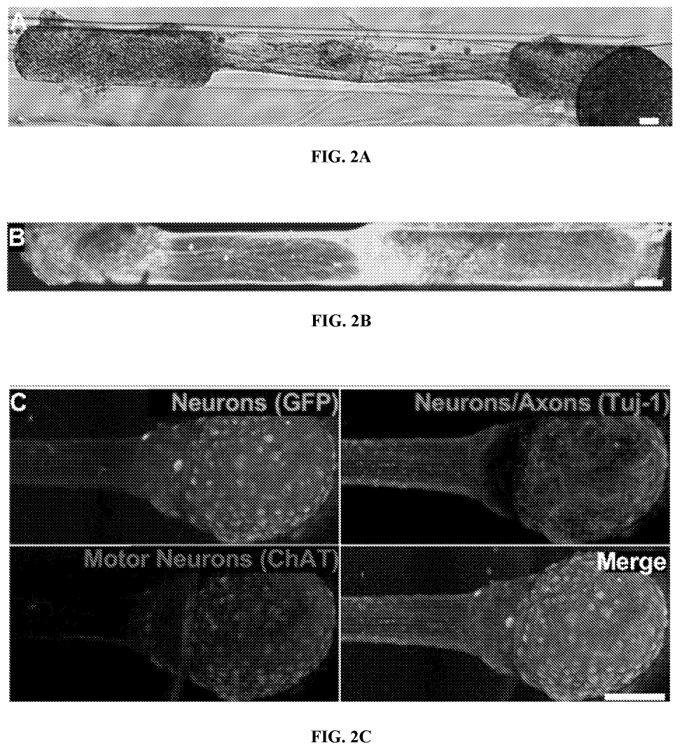

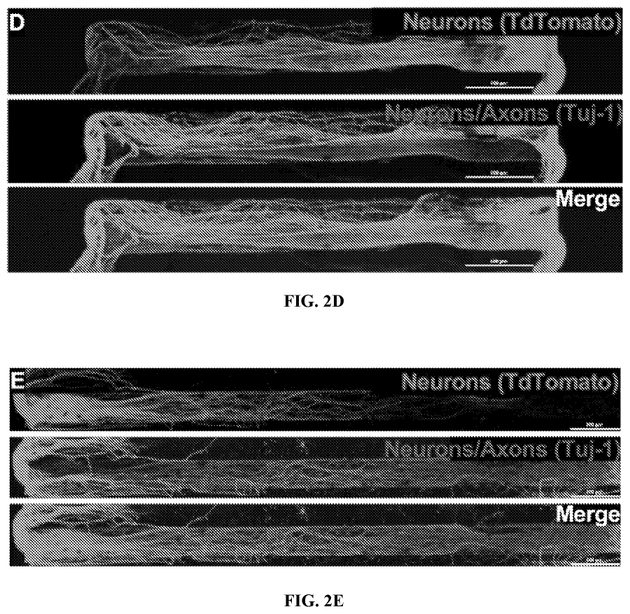

Anatomically-inspired tissue engineered spinal tracts (TE-STs) were fabricated using a modularized microtissue engineering technique previously established by our group. Motor neuron aggregates were seeded inside an agarose microcolumn (701 μm outer diameter and 300 μm inner diameter) with varied lengths spanning 3-12 mm. TE-ST health and viability were visually assessed every other day using phase microscopy. Healthy motor neuron aggregates lacking a necrotic core with dense axonal projections extending through the lumen were readily visualized through the transparent microcolumn (FIG. 2A). Robust survival was confirmed at 7 DIV via a live-dead assay using calcein AM and EthD-1. After incubation, metabolically active cells convert the membrane-permeable calcein AM to calcein, which results in green fluorescence within live cells. Dead cells with a compromised membrane allow for the entry of EthD-1, which binds to nucleic acids, causing red fluorescence. Bright green neurons and axons were readily apparent within the microcolumn with few dead nuclei within the center of the aggregate (FIG. 2B), corroborating the phase microscopy findings.

Immunocytochemistry using choline acetyltransferase (ChAT) and beta-III-tubulin (Tuj1) to discriminate neuronal phenotype (FIG. 2C) confirmed the localization of two discrete populations of ChAT+ motor neurons with Tuj1+ axons extending the length of the micro-column. Although TE-STs could be readily generated at various lengths (FIGS. 2D and 2E), increasing the inter-aggregate distance resulted in slower growth rates (i.e. slower axonal extension in longer microcolumns). In addition, shorter TE-STs had greater axon density and tighter bundling than longer columns at similar time points. While axonal growth rates changed, there was no apparent difference in neuronal health as a function of micro-column length. Collectively, these findings suggest inter-aggregate distance may impact the growth and maturation of constructs in vitro. We found a length of 1.2 cm was necessary to span the injured parenchyma in our previous SCI study. In this previous study using sensory neurons (dorsal root ganglia explants), axonal projections spanned the 1.2 cm micro-column by 14 DIV; however, more slowly growing motor neurons required additional time for axonal growth. Therefore, all TE-STs implanted in subsequent aspects of this study were pre-grown to 1.2 cm in length over 21 DIV.

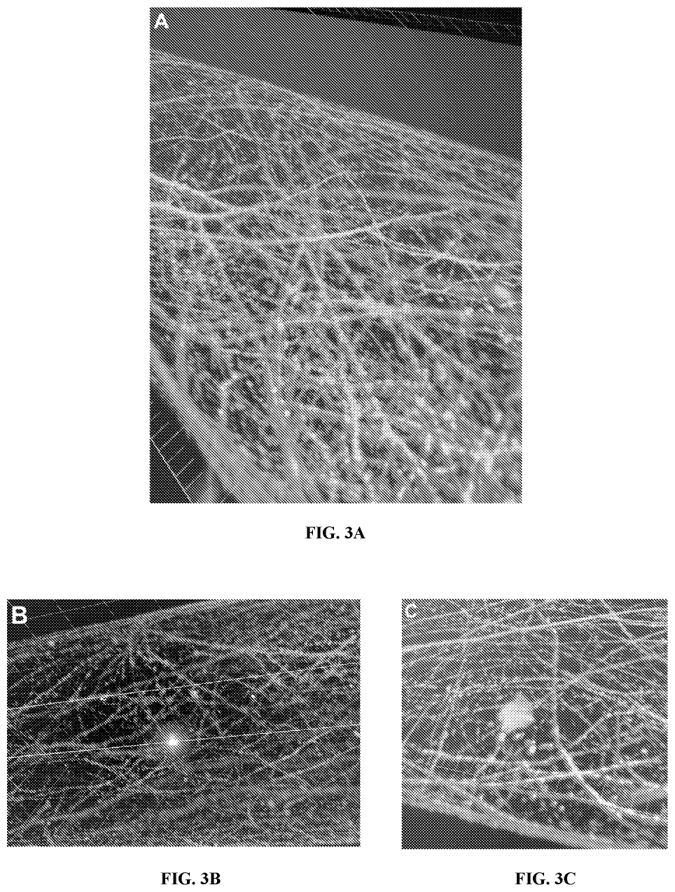

High-resolution imaging of TE-ST axons using a light sheet microscope allowed for visualization of robust networks within the lumen at 21 DIV (FIGS. 3A-3C). Axons spanned the entire length of the lumen and individual fibers could be observed at high magnification. The light sheet microscope acquires images from two objectives and then the software digitally “fuses” the image, which allows greater detail and localization of the fine processes as compared to conventional microscopic imaging. While some axons extended through the collagenous ECM core, a substantial portion grew in a corkscrew fashion on the surface of the ECM at the interface with the agarose micro-column.

Overall, these results demonstrate that TE-STs featuring discrete populations of spinal motor neurons spanned by bundled centimeter-scale axonal tracts can be fabricated by adapting previously established micro-tissue engineering protocols. Moreover, by increasing the time in culture, we can overcome challenges associated with slow axon growth rates without compromising overall health.

### TE-ST Survival Following Transplantation in a Rodent Model of SCI

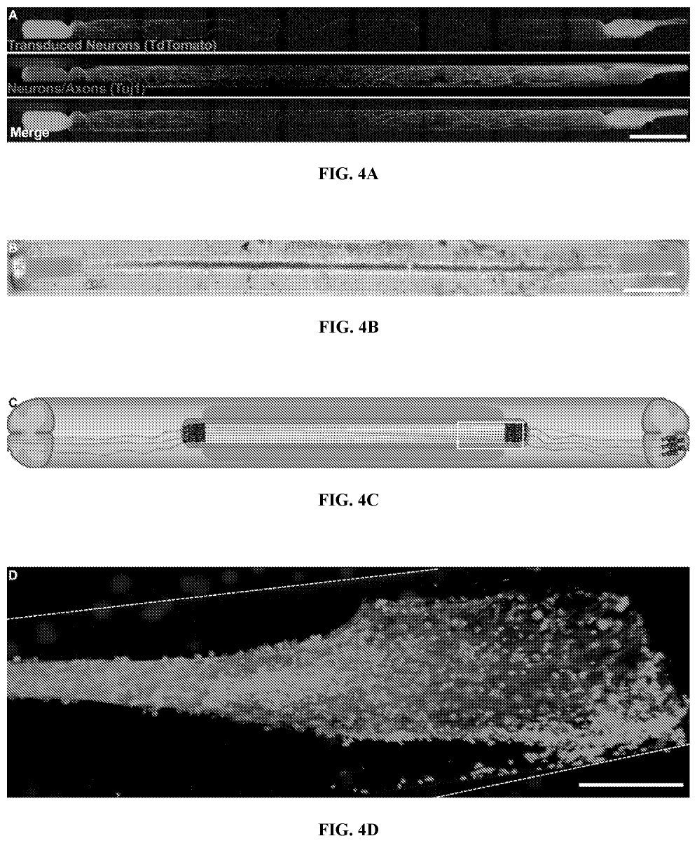

As noted above, prior to transplantation TE-STs were grown for 21 DIV to allow the two populations of motor neurons to extend axons longitudinally within the lumen (FIGS. 4A-4B). TE-STs with healthy motor neurons robustly expressing the red fluorescent marker TdTomato with dense bundled axonal tracts spanning the micro-column length were pre-selected for transplantation into the injured spinal cord (FIG. 4C). Injured spinal cords were optically cleared via refractive index matching to enable the complete visualization of the TE-ST in situ, which allowed interrogation of the survival and architecture of the three-dimensional microtissue at 1 week, 1 month, and 3 months post transplantation. At all time points, TE-STs were visualized within the injured spinal cord following tissue clearing and multiphoton imaging. Agarose micro-columns were found spanning the injury zone that integrated with the host tissue (i.e. host tissue infiltration). Hydrogel degradation was not observed at the time points examined. In some instances, the micro-column followed a slightly curved rather than straight trajectory, although it is unclear if this was a function of implantation or post-mortem tissue processing.

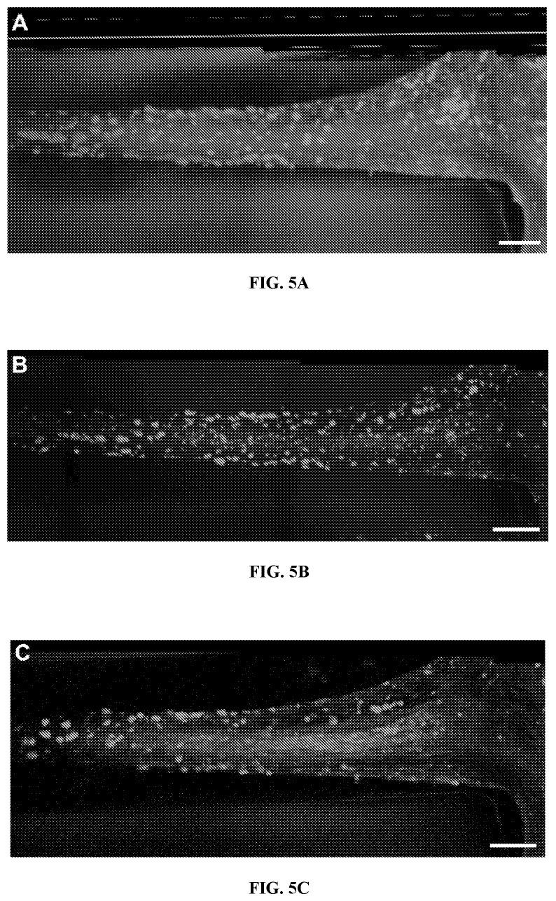

Notably, TE-ST neurons survived transplantation and maintained their preformed cytoarchitecture consisting of discrete neuronal regions spanned by axons at 1 week and 1 month post transplantation (FIG. 4D), suggesting the hydrogel encasement protected the neurons upon delivery and from potentially deleterious effects of the hostile inflammatory post-injury microenvironment. Minimal scar tissue was observed around the construct, indicating the second surgery and transplantation did not result in significant host immunologic response. As expected, modest neuronal attrition within the TE-ST was observed at 3 months post transplantation following volumetric reconstruction (FIG. 5A); however, close analyses of individual z-planes revealed preservation of TE-ST neurons (FIG. 5B) and axon tracts (FIG. 5C) within the lumen. Host-graft integration was evident at this time points, mostly appearing as host cell/tissue infiltration at the ends of the micro-column with only sparse TE-ST outgrowth visualized at 3 months post-implant.

Collectively, these results show that TE-ST neurons survive delivery and maintain their axonal cytoarchitecture following stereotactic implantation to longitudinally span SCI lesions. While promising, the modest neuronal attrition and sparse axonal outgrowth observed at later time points suggest that additional factors may be necessary to improve survival and control integration with host tissue.

Spinal cord injuries affect millions of people worldwide and lead to devastating health, socioeconomic, and psychologic impact to the patients, care takers, and health care systems. Despite decades of research to promote axon regeneration and restore meaningful connectivity within the spinal cord, none have successfully translated into the clinic. Our group—an interdisciplinary team of bioengineers, scientists, and clinicians—have pioneered the development of implantable micro-tissue engineered neural networks for pathway reconstruction following severe neurological injury. Broadly, our goal is to engineer anatomically-inspired pathways in vitro resembling the native architecture that can replace lost populations and their long-distance axonal connections. This present study advances our previous work with the development of tissue engineered spinal tracts (TE-STs) comprised of highly aligned preformed motor tracts designed to reconstruct the damaged spinal cord after injury. Notably, TE-STs survived transplantation and maintained their preformed cytoarchitecture in proof-of-concept experiment using a clinically-relevant epidural balloon-compression rodent model of SCI. While these findings are promising, future detailed mechanistic investigation and optimization will be necessary.

Unlike the peripheral nervous system, the central nervous system has limited intrinsic capacity for regeneration. The main limitation to functional recovery after SCI is that neurons must span from the injury site, across a hostile microenvironment, and then reconnect with appropriate neuronal end targets. Each of these steps have significant challenges. In severe cases resulting in the loss of motor neurons, there is minimal hope for spontaneous recovery due to the inability of the body to replace these cells. In this study, we propose a novel approach to simultaneously replace damaged motor tracts and provide an exogenous source of motor neurons that may replenish the local population lost during the SCI pathologic sequalae. Ultimately, pathway reconstruction using TE-STs would both replace lost axonal circuits and integrate with the local spinal neuronal populations. Careful delivery of the TE-ST spanning the injured spinal cord allows for the transplanted motor neurons to interact with the spared host tissue (both rostral and caudal to the injury zone) and restore connectivity across the lesion. Additionally, the hydrogel encasement protects the exogenous neurons from the hostile microenvironment of the injured spinal cord. Collectively, this strategy may provide a functional axonal bridge through the glial scar and across the injury zone.

Our understanding of spinal cord regeneration has significantly progressed with advanced investigative tools, such as high resolution microscopy and optogenetics. There is ample evidence within the literature that existing circuitry within the spinal cord functions as a neuronal relay following SCI, which may be a mechanism exploited in the future by TE-STs. For instance, there is a significant interest in propriospinal neurons (PSNs), a special class of spinal interneurons with projections to other spinal segments, between dorsal and ventral horns, as well as bilateral connections between the left and right side. Functionally, PSNs influence and modulate inputs from ascending or descending pathways, such as motor commands from the brain, and have a significant role in generating locomotion activity in the spinal cord. Large populations of PSNs are found in the cervical and lumbar regions of the spinal cord with long-spanning axons connecting the two regions. In combination, it is thought that PSNs are responsible for fine motor coordination by providing an electrical “copy” of the activation pattern from the brain to other segments of the spinal cord not being directly activated by the brain. It has been suggested that PSNs have a significant role in functional recovery after SCI by rapidly crossing an injury site and reestablishing synaptic connections with ventral spinal motor neurons. Functional recovery after SCI has shown that axonal sprouting from cortical spinal tracts will preferentially synapse with PSNs rather than re-establish previous long-distance connections to lower order motor neurons, essentially utilizing PSNs as a relay between the brain and lower order neurons.

A number of potentially neuroprotective and/or neuroregenerative agents have been evaluated post-SCI. Neuroprotective agents attempt to reduce the secondary damage due to attenuating cell death and/or modulating inflammation and glial scar formation. For instance, methylprednisolone was a prominent neuroprotective agent deployed during the acute phase of SCI but its use is controversial due to the absence of convincing and significant neurological improvement as well as harmful side effects. Other pharmacological agents have shown promise in animal models of SCI, but clinical efficacy and safety have not been demonstrated. Moreover, there is a growing body of evidence that suggests some aspects of neuroinflammation following injury may be neuroprotective and that more novel immunotherapeutics are needed to guide the immune response to a more favorable wound-healing response. Neuroregenerative treatments attempt to enhance the endogenous regeneration process and to alter barriers to intrinsic regeneration. Investigational efforts involve interventional strategies and approaches, including agents to break down inhibitory factors (e.g., chondroitinase), biomaterials as regenerative scaffolds, growth factors to stimulate axonal extension, and/or cell-based approaches to modulate secondary pathology or replace lost cells. Of these approaches, cell-based therapies have made significant advancements for treatment of SCI in preclinical models. For example, oligodendrocytes and Schwann cells have been well-documented to improve regeneration and ameliorate the injury cascade following spinal cord trauma; however, functional recovery remains limited if neurons are damaged or axons have to span long distances. Neural stem/progenitor cells (NSPCs) are an exciting cell source for transplantation because they can differentiate in vivo into neurons or glial cells and have limited risk for tumorigenesis compared to pluripotent stem cells. Recent work by the Tuszynski group have shown that NSPCs grafted into an injured spinal cord integrate with the cortical spinal tract and the graft can elicit local neuronal activity in the host tissue. While cell therapies may have some utility, clinical implementation remains limited due to challenges associated with cell survival, batch-to-batch variability, and scale up. To address these concerns, some groups have bioengineered scaffolds seeded with cells to improve survival and integration following transplantation. However, despite promising data in animal models, those that have progressed to human clinical study have yet to demonstrate meaningful and consistent improvements in functional recovery. These challenges in translating into clinical efficacy appear to be strongly influenced by an inability to overcome the inhibitory environment of the glial scar and to promote/guide long-distance axon re-growth in humans. Moreover, these strategies often underappreciate the role of the native architecture.

Our approach to address the significant challenges associated with SCI (i.e. the loss of neurons and diminished capacity for long-distance regeneration across a hostile microenvironment) is to use structured engineered microtissue with a design that is biomimetic of endogenous anatomy and functional characteristics. Moreover, these preformed tissue engineered medical products have a defined final structure, health and potency that can be assessed pre-implant to reduce variability. Further, tissue engineered constructs are scalable to even larger human lesions as shown by our ability to grow implantable axon-based tissue several centimeters in length by leveraging traditional axonal growth within microcolumns or by exploiting axon “stretch-growth” in custom mechanobioreactors. While our strategy for pathway reconstruction is designed to the address major challenges in SCI repair, significant future efforts are required to validate and optimize this paradigm.

To test the efficacy of TE-STs, we utilized a previously established balloon compression-induced SCI model, which recapitulates key features associated with common clinical scenarios, such as motor vehicle accidents, sports-injuries, and falls. Although transection and hemostatic clip compression models are commonly used in research, SCI in humans typically presents as a contusion or compression injury rather than complete transection. Similar to the transection or evacuation models, the balloon compression model reliably leads to neuronal loss of the grey matter and (partial or complete) axonal disconnection of the white matter tracts spanning the injury site. These advantages make the balloon compression-induced SCI model the preferred choice to study the efficacy of bridging healthy spinal cord tissue with TE-ST across long-spanning SCI. In addition, traditional contusion models require a laminectomy at the level of the cord to induce the injury; in contrast, our model preserves the integrity of the spinal canal after injury by performing a laminectomy at a caudal level to introduce the catheter, which can be advanced through the canal to induce the injury at the desired level. This advantage eliminates the decompression variable from the experiments and makes the injury model less invasive. Previously, we performed histological as well as functional assessments to test the reliability of balloon-induced compression model, and notably we found injured spinal cords appeared to have similar features to postmortem human cord samples. All animals display a significant loss of hind limb function that partially recovers within the 3-4 week period post-injury and then plateaus, offering a flexible time window for experimental intervention. In our experience, performing balloon compression via lower laminectomy at L3-4 protects the cord from undesirable surgical injury. This minimally invasive technique also reduces scar tissue formation compared to other models (transection and compression), which facilitates re-exposure for delayed repair and lowers the risk of wound complications, such as dehiscence, hemorrhage and infection. Finally, balloon compression does not cause direct injury to the spinal canal vasculature which leads to significantly lower blood loss. However, one major limitation compared to other models is the balloon-compression followed by delayed repair is much lower throughput than other models and requires significant training and experience.

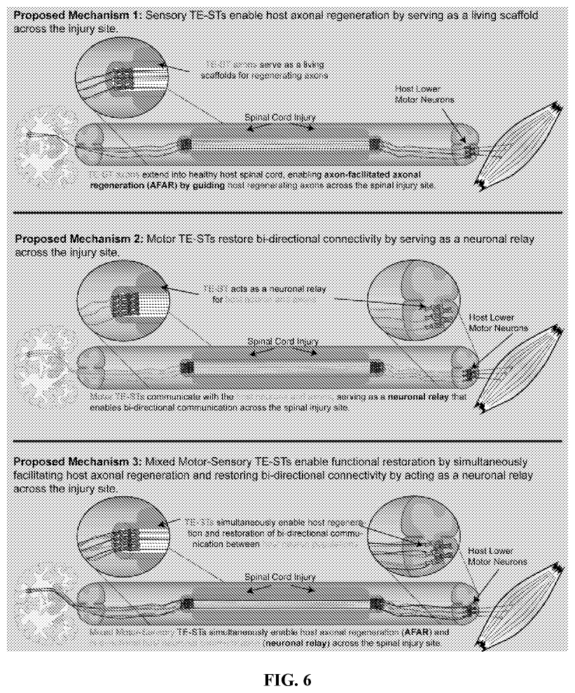

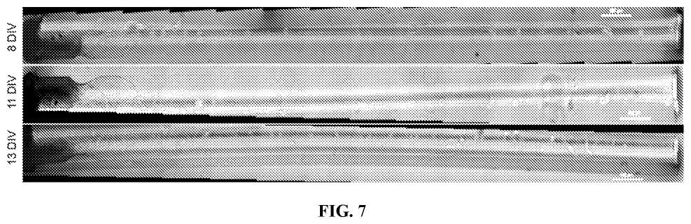

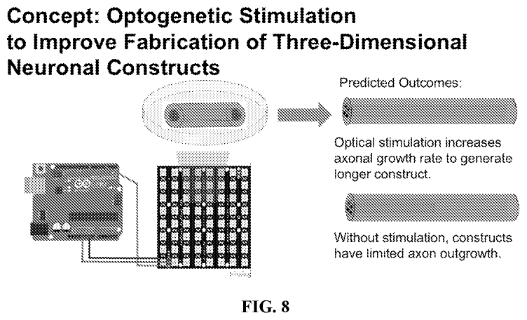

In the current study, we demonstrated that novel TE-STs containing allogenic embryonic motor neurons survive transplantation and maintain their preformed cytoarchitecture of internalized axonal tracts. Additional optimization will be necessary to improve motor neuron viability and integration with host tissue at chronic time points. In this study, our ECM cocktail (collagen+laminin) was optimized for in vitro growth; future studies will examine strategies for sustained release of growth factors either in the ECM or within the agarose microcolumn to promote neuronal survival, axon extension, and potentially modulate the inflammatory microenvironment following transplantation. Given future optimization of TE-ST survival and integration, upcoming studies will explore the potentially multi-faceted mechanisms-of-action whereby TE-STs may facilitate spinal cord regeneration and functional recovery following SCI. For instance, we predict that TE-STs may serve as an axon-based living scaffold to facilitate host axonal regeneration via the AFAR mechanism, serve as a synaptic-based neuronal-axonal relay that reconnects neural circuitry across the lesion zone, or a combination of both of these mechanisms acting simultaneously based on the phenotype(s) of the neurons present within the TE-STs (FIG. 6). To elucidate the relative contributions of these putative mechanisms-of-action, additional techniques such as cortical tract tracing and PSN tracing will be necessary as well transsynaptic tracing and optogenetics experiments.

### SUMMARY

Our team has developed a novel implantable tissue engineered spinal tract for intraspinal neuronal replacement and axon tract reconstruction following SCI. We report fabrication parameters and in vitro characterization as well as proof-of-concept testing in a clinically-relevant rodent model of SCI. In this study, a 1.2 cm TE-ST comprised of two discrete populations of allogenic embryonic motor neurons with axons spanning the lumen survived for up to 3 months post transplantation. Future work is necessary to improve survival and outgrowth, as well as to assess the potential efficacy of this strategy in promoting behavioral recovery.

### Example 2—Optogenetic Experiment

Optogenetic stimulation is a method to rapidly increase sensory neurite outgrowth in 2D. Unlike other explored methods of increasing the rate of axonal growth, such as electrical stimulation, optogenetic stimulation provides cell-type specific, temporally precise excitatory and inhibitory control of neural activity in genetically distinct cell populations. Such a phenomenon is achieved through the use of channelrhodopsins, light-gated ion channels that can be introduced to and expressed by desired cell populations. Previously, it has been shown to promote the secretion of growth factors in dorsal root ganglion and result in enhanced axonal outgrowth. This Example describes the development of a method to stimulate light-sensitive spinal motor neurons.

Initially, as a baseline, motor neurons were plated on one end of a 1 cm agarose micro-column containing collagen and laminin. The transparent agarose micro-column allows for imaging over time using phase microscopy. As illustrated in FIG. 7, without light stimulation, motor axons slowly extended into the column and were unable to reach more than a couple millimeters by 13 days in vitro. These findings suggest that despite the presence of collagen ECM, motor neurite extension slows down over time, suggesting neurite outgrowth may be limited by intrinsic mechanisms. To enhance axonal growth during neuronal construct fabrication, the method described herein includes first plating light-sensitive neurons transduced to express channelrhodopsin in an agarose microcolumn. The transparent agarose hydrogel allows for light application to reach the neurons during culture. As shown in the schematic of FIG. 8, light-sensitive neurons plated in a micro-column can then be stimulated with light emitted from an LED to enhance axonal growth during the fabrication process.

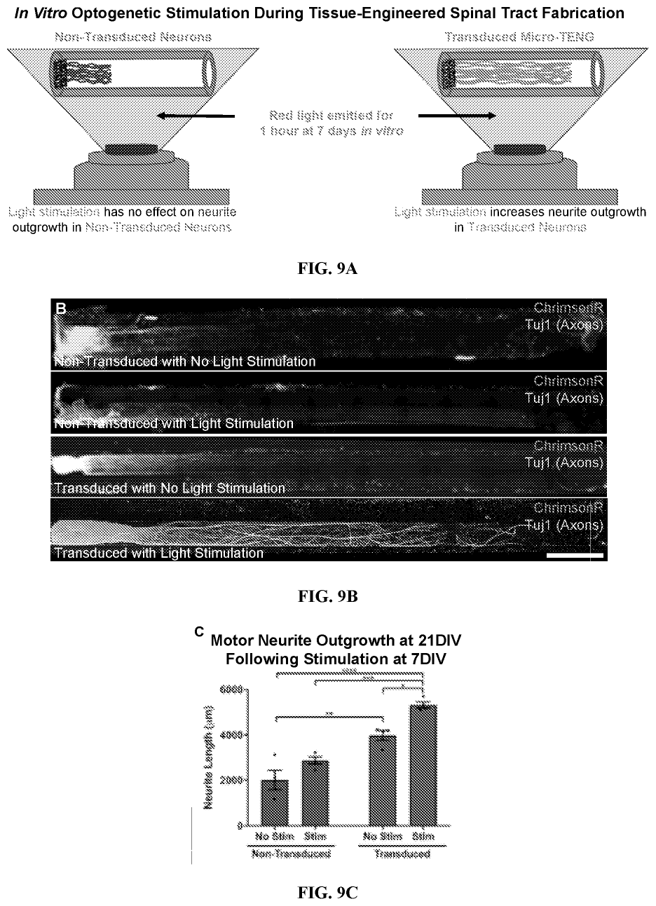

The effect of optogenetic stimulation on growth was examined using non-transduced and transduced neurons with and without light stimulation (FIGS. 9A-9C). More specifically, motor micro-TENGs were transduced to express chrimsonR, a channelrhodopsin, tagged with tdTomato for visualization (red). Three-dimensional motor neurite growth was assessed at 21 DIV after a single round of stimulation at 7 DIV using a custom-made LED. Control constructs were exposed to the same conditions as experimental constructs, but did not receive LED stimulation. Greater neurite outgrowth was found following optogenetic stimulation of transduced micro-TENGs compared to non-stimulated transduced micro-TENGs (p<0.05), or non-transduced micro-TENGs with (p<0.001) or without stimulation (p<0.0001).

Notably, motor axon growth in the transduced, light-stimulated group reached 5.3 mm by 21 DIV or a 2.3-fold increase compared to the non-transduced, non-stimulated group. Interestingly, there appeared to be a slight increase in motor outgrowth in the non-stimulated transduced group compared to the other non-transduced groups. These findings suggest optogenetic stimulation may be useful as an advanced biofabrication paradigm to generate tissue engineered neuronal constructs with longer spanning axons. Since axon pathfinding is highly dependent on structural cues from other axons and/or trophic factors secreted by other cells, this study tested the effect of optogenetic stimulation on unidirectional motor neurite outgrowth (i.e. only one neuron population) because the rate of neurite outgrowth would likely be influenced by axons extending from an opposite side. These findings suggest that in vitro light stimulation enhances the fabrication process of tissue engineered neuronal constructs.

Since light activation has been reported to impact nerve regeneration potentially via temperature-sensitive proteins channels, a simulated stimulation paradigm was also used to measure temperature over time. Media dishes were illuminated from a custom light-emitting diode for 1 hour with a frequency of 10 Hz. Temperature was sampled every 15 minutes starting at baseline (illuminated: 35.4° C.±0.1° C.; control: 35.3° C.±0.2° C.), 15 minutes (illuminated: 36.4° C.±0.3° C.; control: 36° C.±0° C.), 30 minutes (illuminated: 36.8° C.±0.3° C.; control: 36.1° C.±0.1° C.), and 60 minutes (illuminated: 37° C.±0.5° C.; control: 36.3° C.±0.1° C.) (Table 1). Although illumination slightly increased the temperature over time, two-way ANOVA did not reveal any significant difference over time or across groups, suggesting differences in temperature did not impact growth. Data is shown as mean±standard deviation.

TABLE 1
Effect of light stimulation on media temperature.

| Conditions | Baseline | 15 min | 30 min | 60 min |
|---|---|---|---|---|
| Illuminated | 35.4° C. ± | 36.4° C. ± | 36.8° C. ± | 37° C. ± |
| (n = 3) | 0.1° C. | 0.3° C. | 0.3° C. | 0.5° C.; |
| Control | 35.3° C. ± | 36° C. ± | 36.1° C. ± | 36.3° C. ± |
| (n = 3) | 0.2° C. | 0° C. | 0.1° C. | 0.1° C. |

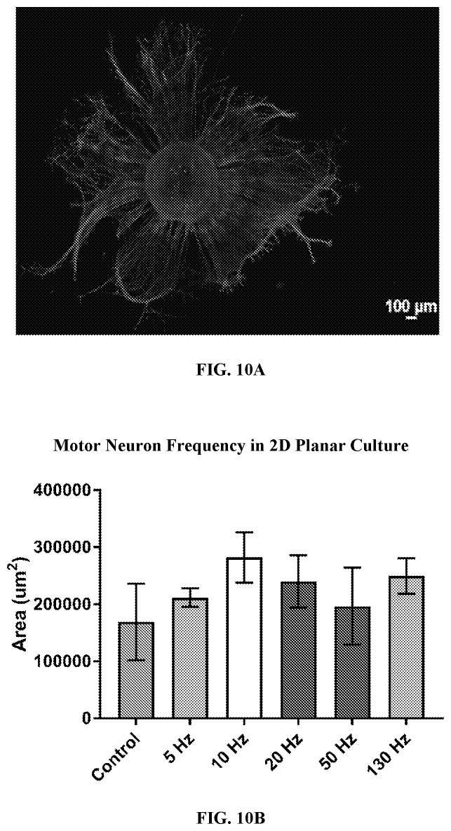

Next, the effects of different frequencies on neurite outgrowth were examined (FIGS. 10A-10B). A planar culture of embryonic rodent motor neurons were transduced to express a channel rhodopsin. At 4 days in vitro, light-sensitive neurons were stimulated using a custom light-emitting diode for one hour at various frequencies (0, 5, 10, 20, 50, and 130 Hz). Cultures were fixed at 14 days in vitro and neurites were labeled with Tuj1 (purple) (FIG. 10A). Transduced motor neurons were also visualized by red fluorescent reporter protein (TdTomato). Confocal images were quantified for total area of neurite outgrowth (FIG. 10B), with neurite area calculated using a custom MATLAB script. The highest level of neurite outgrowth was seen at 10 Hz, which is similar to what was observed for in vivo optogenetic stimulation. Interestingly, it was observed that motor neurons subjected to stimulation above a certain intensity threshold in stimulation experiments showed signs of neuron death, such as axonal beading. However, using the protocol that did not result in any changes in temperature, differences in neurite outgrowth between non-transduced stimulated groups and the non-transduced non-stimulated groups were observed, suggesting that stimulation may also have harmful effects on non-transduced aggregates.

### Example 3—Aggregate Transduction and Engineered Spinal Tract Transplantation

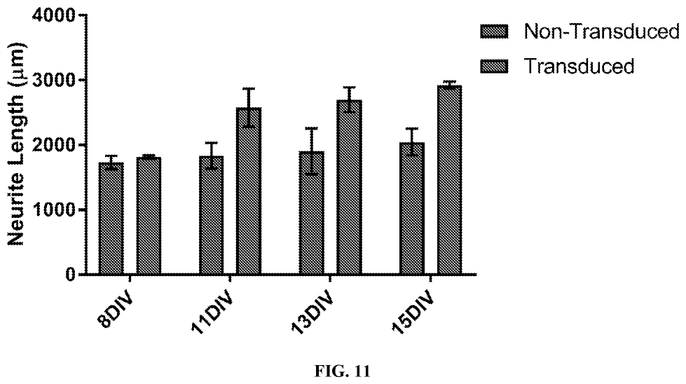

Referring to FIG. 11, embryonic motor neurons were aggregated into discrete populations of cells and then cultured in agarose micro-columns. Motor neurites extended from the discrete population of neurons, resembling the in vivo spinal cord architecture. At 7 days in vitro, engineered spinal motor tracts were exposed to light for one hour with a frequency of 10 Hz. Although no differences were detected in neurite length at 8 days in vitro (i.e. 1 day after stimulation), there was a stark increase in growth by day 11 compared to wild-type (non-transduced cultures), which continued for the remaining days in culture. These findings suggest that one dose of light stimulation may have accelerated axonal extension in vitro by activating slow-acting downstream genetic and molecular changes.

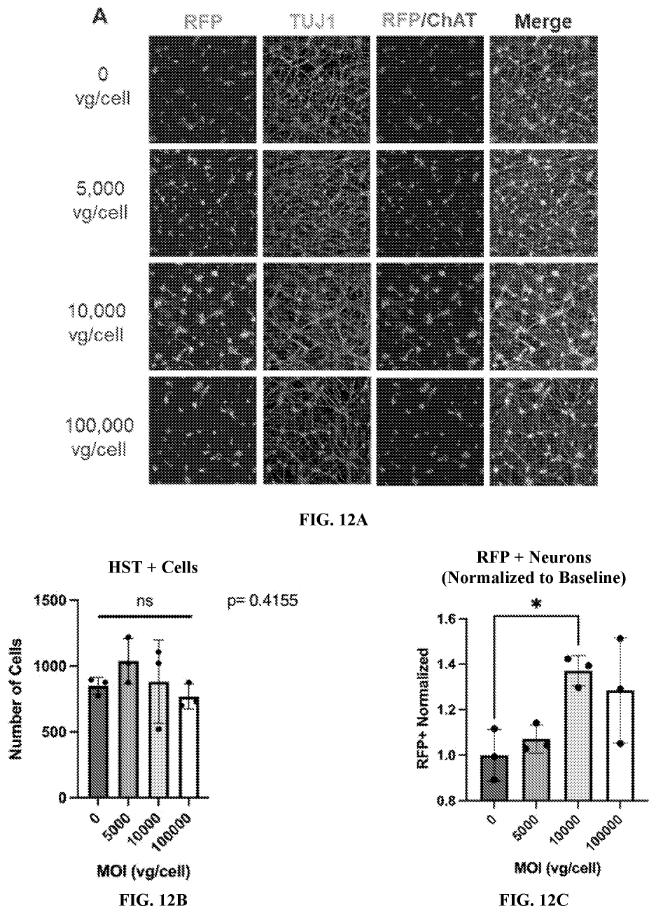

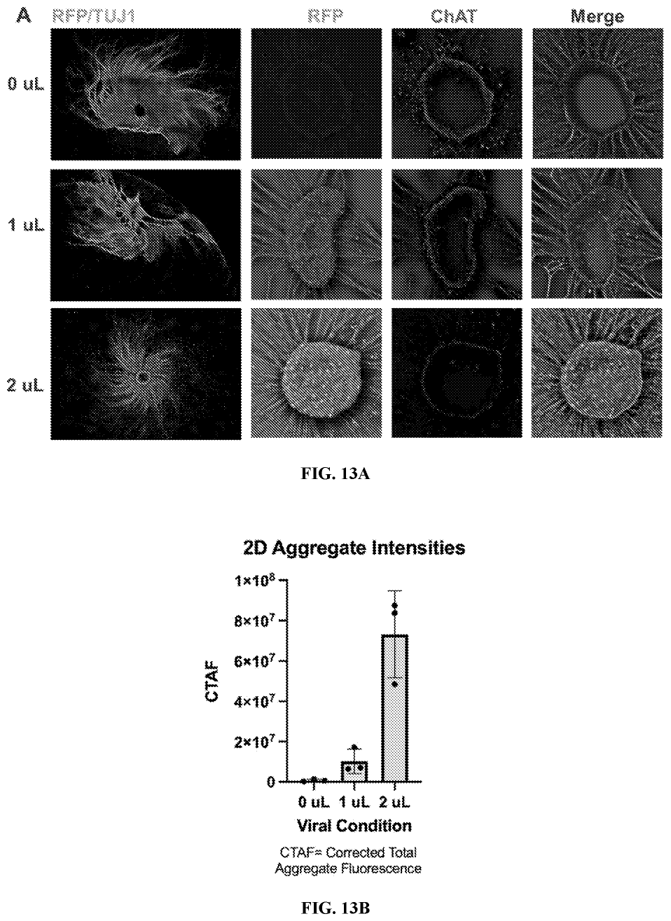

Next, the effects of vector genome per cell (vg/cell) levels were examined in a two-dimensional culture of human iPSC-derived motor neurons transduced with varying amounts of AAV8-hSyn-ChR2-tdTomato (FIGS. 12A-12C). At 12 days in vitro, no differences in total number of cells labeled with hoechst nuclear marker (HST) were detected; however, more transduced neurons labeled with anti-red fluorescent protein (RFP) were observed following transduction at 10,000 vg/cell. The effects of viral dosing on human motor neuron aggregate transduction were also examined, as illustrated in FIGS. 13A-13B. Motor neurons were transduced during the aggregation process, and viral dosing was examined for human iPSC-derived motor neurons after treatment with AAV1-hSyn-ChrmR-tdTomato was established based on cytotoxicity and corrected total fluorescence measured from the aggregate (CTAF). At 12 days in vitro, greater aggregate fluorescence was detected following treatment with higher viral dose. These findings suggest optogenetic viral transduction of three dimensional human motor neuron aggregates is comparable to embryonic rodent motor neurons.

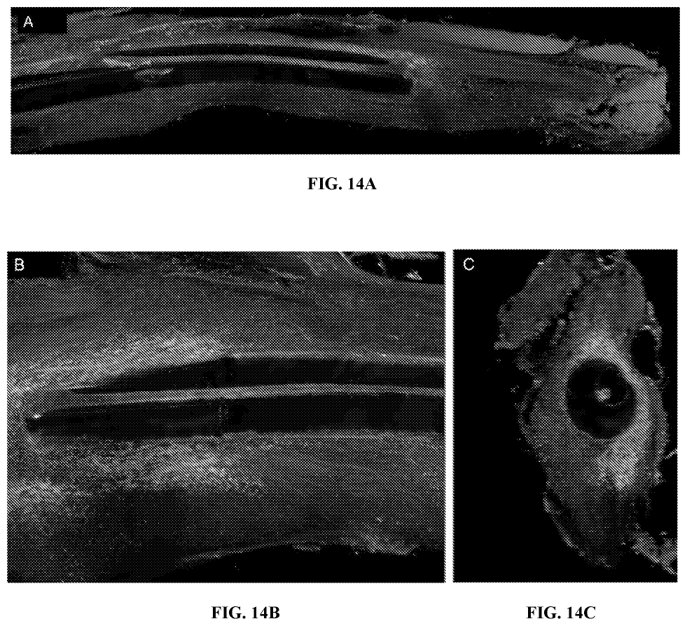

Finally, engineered spinal tracts were studied following spinal cord injury (FIGS. 14A-14C). Engineered spinal tracts were fabricated in vitro for 14-21 days and then transplanted at 14 days post spinal cord injury. At 7 days, 1 month, and 3 months after injury, spinal cords were harvested and then optically cleared to enable visualization of the three dimensional architecture using light sheet microscopy. As illustrated in FIG. 14A, the engineered axonal cytoarchitecture in the protective agarose microcolumn spanned the injury site, with neurons are visible at the end the microcolumn. Neurite outgrowth into spinal cord parenchymal tissue was also observed, as seen in FIG. 14B. FIG. 14C further shows an axial rendering of the micro-column showing the bundle of axons in the center of the lumen protected by the agarose micro-column. This data suggests that axons originating from the engineered spinal tracts extended into the uninjured spinal cord parenchyma. Therefore, without wishing to be bound by theory, it is believed that in vivo optogenetic activation of engineered microtissue may further enhance neurite outgrowth or modulate in vivo neuronal activity.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of fabricating a tissue engineered spinal tract, the method comprising:
(a) seeding a plurality of optogenetic neural cells at each end of a hydrogel microcolumn to form a construct; and
(b) culturing the construct in vitro while stimulating the plurality of optogenetic neural cells with a predetermined wavelength of light.

2. The method of claim 1, wherein the plurality of optogenetic neural cells are transduced with one or more transgenes.

3. The method of claim 2, wherein the one or more transgenes include channelrhodopsin, Channelrhodopsin-2, ChrimsonR, CatCh, a halorhodopsin, a archaerhodopsin, an optogenetic sensor for calcium, an optogenetic sensor for chloride, and/or an optogenetic sensor for membrane voltage.

4. The method of claim 2, wherein the one or more transgenes include channelrhodopsin, Channelrhodopsin-2, ChrimsonR, CatCh, a halorhodopsin, and/or a archaerhodopsin.

5. The method of claim 2, wherein the optogenetic sensors for calcium include Aequorin, Cameleon, or GCaMP.

6. The method of claim 2, wherein the optogenetic sensors for chloride include clomeleon.

7. The method of claim 2, wherein the optogenetic sensors for membrane voltage include Mermaid.

8. The method of claim 2, wherein the plurality of optogenetic neural cells are transduced using AAV to insert ChrimsonR with a human synapsin promoter.

9. The method of claim 1, wherein the construct is a biocompatible construct.

10. The method of claim 1, wherein the construct is an implantable construct.

11. The method of claim 1, further comprising:
(c) determining axons growth from the plurality of neural cells has reached a particular length; and
(d) responsive to the particular length of axon growth being determined to have been reached, packaging and/or providing the micro-column for implantation.

12. The method of claim 11, wherein the particular length is a predetermined desired length.

13. The method of claim 11, wherein the particular length ranges from about 0.5 to about 5 centimeters.

14. The method of claim 11, wherein the particular length ranges is about 1.2 centimeters.

15. The method of claim 11, wherein step (c) comprises imaging the micro-columns and neural cells therein.

16. The method of claim 1, wherein the plurality of optogenetic neural cells with which the micro-column is seeded at step (a) comprise a population of neural cells.

17. The method of claim 16, wherein the population of neural cells is seeded individually, as an organoid, or as an aggregate.

18. The method of claim 17, wherein the neural cell aggregate comprises a plurality of approximately spherical aggregates of neural cells.

19. The method of claim 17, wherein each neural cell aggregate comprises cells at a density ranging from about 10,000 to about 3,000,000 neurons per aggregate.

20. The method of claim 19, wherein each neural aggregate comprises cells at a density ranging from about 40,000 to about 65,000 motor neurons per aggregate.

21. The method of claim 17, wherein a plurality of the neural cell aggregates exhibit a diameter of between 10 μm to 2500 μm.

22. The method of claim 21, wherein the diameter of the neural aggregate is about 500 μm.

23. The method of claim 1, wherein the micro-column comprises a hydrogel sheath and a core comprising an extracellular matrix (ECM), and wherein the neural cells are seeded to be in direct contact with the ECM of the core.

24. The method of claim 23, wherein the hydrogel includes agarose, gelatin, silk, chitosan, hyaluronic acid, methylacrylated gelatin, methylacrylated hyaluronic acid (MeHA), or combinations thereof.

25. The method of claim 23, wherein the hydrogel sheath comprises methylacrylated hyaluronic acid (MeHA).

26. The method of claim 23, wherein the hydrogel sheath comprises agarose and gelatin.

27. A method of treating a spinal cord injury in a subject, comprising contacting a lesion in the spine of the subject with a tissue engineered spinal tract made by the process of claim 1.
